(19) United States
(12) Patent Application Publication
(10) Pub. No.: US 2024/0226333 A1
(43) Pub. Date: Jul. 11, 2024

(54) NEW PROMOTER SEQUENCE FOR GENE THERAPY

(71) Applicant: UNIVERSITÁ DEGLI STUDI DI PADOVA, Padova (IT)

(72) Inventors: Alessandra BIFFI, Padova (IT); Valentina POLETTI, Padova (IT)

(73) Assignee: UNIVERSITÁ DEGLI STUDI DI PADOVA, Padova (IT)

(21) Appl. No.: 18/559,280

(22) PCT Filed: May 3, 2022
(86) PCT No.: PCT/IB2022/054069

§ 371 Date: Nov. 6, 2023

(30) Foreign Application Priority Data
May 6, 2021 (IT) 102021000011576

(51) Int. Cl. A61K 48/00; C07K 14/74; C12N 15/86

(52) U.S. Cl. CPC A61K 48/0058; C07K 14/70539; C12N 15/86; C12N 2740/15043; C12N 2830/008

(57) ABSTRACT

The present invention relates to a promoter sequence for efficient and sufficient expression of transgenes as well as to gene transfer vectors comprising said promoter sequence for use in therapy. In particular, the present invention relates to lentiviral vectors that provide gene therapy for pathological conditions of the central nervous system.

### TECHNICAL FIELD

The present invention relates to a promoter sequence for efficient and sufficient expression of transgenes as well as to gene transfer vectors comprising said promoter sequence for use in gene therapy. In particular, the present invention relates to lentiviral vectors that provide gene therapy for pathological conditions of the central nervous system.

### Background

Neuroinflammation is characterized, in addition to other phenomena, by microglial cell-activation that plays a major role in the pathogenesis and progression of several inherited and acquired neurodegenerative diseases, including Lysosomal Storage Disorders (LSDs). The beneficial effect of ex vivo gene therapy (GT) based on the use of genetically modified hematopoietic stem cells (HSCs) for these conditions relies on microglia-functionally equivalent cells derived from the transplanted genetically modified HSCs engraft into the recipient's Central Nervous System (CNS) where they exert homeostatic and scavenging functions, normalize microglia cell homeostasis and reduce neuroinflammation after the treatment. Moreover, in HSC GT for LSDs, enzyme-competent, genetically corrected, transplant derived microglia-like cells release the therapeutic factor (the defective lysosomal enzyme of interest for each LSD) to be available for all the resident CNS cells. Similarly, microglia-mediated delivery of immunomodulatory or other antioxidant factors could be explored to mitigate neuroinflammation and neurodegeneration.

An ideal lentiviral vector (LV) for this GT strategy for neurodegenerative diseases should drive a regulated expression of the therapeutic transgene in the HSPC-derived microglia-like cells: upregulated in the presence of neuroinflammation and downregulated under homeostatic CNS conditions. Such transcriptional pattern is typical of the human HLA-DRA gene, encoding the Class II Major Histocompatibility Complex (MHC-II) α-chain immunoglobulin domain of Histocompatibility Leukocyte Antigen. Class II molecules are constitutively expressed at a low basal level by professional antigen presenting cells, as microglia cells, and their expression is strongly upregulated during inflammation (Jenny Pan-Yun Ting et al. Cell 2002).

However, there remains a need to develop promoter sequences, gene transfer vectors, retroviral, particularly lentiviral vectors, which allow efficient and sufficient expression of transgenes.

### SUMMARY OF THE INVENTION

The technical problem posed and solved by the present invention is to provide a promoter sequence that allows for efficient control of the expression of transgenes, particularly in immune cell types, such as, relevant CNS-associated myeloid/microglia-like cells derived from HSC transplantation. Such a problem is solved by a promoter sequence according to claim 1 of the present invention.

Following an extensive analysis of the putative regulatory region of the endogenous human HLA-DRA gene, the authors of the present invention have particularly designed a synthetic promoter aimed at having a minimal basal transcriptional activity and at being highly inducible upon microglia cell activation. To disclose the regulatory elements that control this transcriptional pattern, the inventors have analysed the putative regulatory region of the human HLA-DRA gene by integrating publicly available transcriptional and epigenetic data produced by the Broad Institute-Encode Project in human hematopoietic (K562 cells) and non-hematopoietic cell lines. By a deep in silico analysis of the genomic region upstream and downstream to the human HLA-DRA transcriptional start site, they have identified regions epigenetically marked as active promoters and active enhancers, bound by hematopoietic-specific transcription factors as indicated by Chip-seq genomic maps. The epigenetically identified promoter region was confirmed by RNA-seq data.

Once identified a broad putative promoter region, the inventors have refined it by searching for general (TATA box) and specific DNA motifs (Beresford G. W, Nat Immunol 2001) known to regulate Class II gene expression, as: i) a W/S- and X1-boxes, which serve as binding site for the multicomponent transcription factor RFX that recruits the principal MHC II master regulator, i.e. the Class II Transcriptional Activator (CIITA); ii) a X2- and Y-boxes, which act as binding sites for X2BP and NF-Y transcription factors.

This in silico analysis was performed with a motif-based sequence analysis software (The MEME Suite, Bailey T L et al., Nucleic Acids Res. 2015), using as a reference the S-, X- and Y-box sequences published in Beresford et al (Beresford G. W, Nat Immunol 2001). A putative HLA-DRA core promoter region was thus identified and adapted to lentiviral system to generate a final synthetic HLA-DRA promoter (hereafter, hHLA). The HLA-DRA-based promoter of the invention is hence designed with a compact size, suitable for a lentiviral system.

As clearly demonstrated by the results reported in the experimental section of the present specification, the novel hHLA promoter designed and developed by the authors of the present invention, when inserted in a lentiviral vector to control the expression of a given transgene in immune-cell types, is able to induce gene expression, at mRNA and protein level, in vitro and in vivo, also in relevant CNS-associated myeloid/microglia-like cells derived from HSC transplantation.

In one embodiment, the promoter sequence of the invention is synthetized and cloned in a standard LV backbone to control the expression of a biologically inactive reporter gene, the Green Fluorescent Protein (GFP), to create a reporter LV. Such vector is currently under preclinical investigation and demonstrated the capability to be produced at very high infectious titer, to efficiently and safely transduce human cell lines and primary Hematopoietic Stem/Progenitor Cells (HSPCs) in vitro, and to transduce and drive a high transgene expression levels in vivo in long-term repopulating HSCs in a transplantation assay performed in wild-type mice.

The capability of the promoter sequence of the invention to respond to immune cell-activation increasing its transcriptional level is currently under investigation in human microglia cells. Notably, the new hHLA promoter of the invention can induce a high transgene expression in vivo, in myeloid/microglia cells derived from transplanted HSPCs, suggesting its capability to maintain the correct regulatory profile upon pluripotent-cell differentiation to microglia cells, not being subjected to epigenetic silencing or other forms of undesired regulation.

This type of promoter can be strongly beneficial for cell and gene therapy approaches requiring a basal level of expression of therapeutic proteins that increase in the presence of neuroinflammation, a pathological status typical of multiple inherited or acquired severe neurodegenerative diseases of childhood and adulthood.

The results obtained by the authors of the present invention hence strongly support the usage of this new hHLA promoter for therapeutic purposes, to determine a regulated expression of therapeutic or immune-modulatory molecules in gene and cell therapy approaches to treat pathological conditions of the CNS, particularly for those characterized by neuroinflammation.

It forms hence part of the present invention:
A promoter sequence comprising or consisting of a polynucleotide sequence having SEQ ID NO:1 or that shares more than 90%, preferably more than 95%, more preferably more than 99% identity with the sequence of SEQ ID NO: 1;
A gene transfer vector, in particular a lentiviral vector, comprising the promoter sequence according to any of the embodiments as defined in the present specification and in claim 1;
A viral vector particle comprising the promoter sequence or the vector according to any of the embodiments as defined in the present specification and in the claims;
An isolated host cell comprising the promoter sequence, or the vector, or the viral vector particle according to any of the embodiments as defined in the present specification and in the claims;
An isolated host cell infected or transduced with the gene transfer vector or particle according to any of the embodiments as defined in the present specification and in the claims;
A pharmaceutical composition comprising the gene transfer vector, or particle, or the cell according to any of the embodiments as defined in the present specification and in the claims and one or more excipients or diluents or carriers;
A gene transfer vector, a particle, a pharmaceutical composition, a cell according to any of the embodiments as defined in the present specification and in the claims for use as a medicament or vaccine, for use in a gene therapy, for use in hematopoietic stem therapy, and/or for treating a pathological condition of the CNS, in particular a pathological condition of the CNS characterized by neuroinflammation;
Use in vitro of a gene transfer vector, or a particle according to any of the embodiments defined in the present specification and in the claims, for regulating the expression of a transgene in a host cell, in particular in a hematopoietic stem cell or a hematopoietic progenitor cell.

Preferred features of the present invention are the object of the dependent claims. Additional features and advantages of the various aspects of the present invention will become apparent from the following detailed description of its preferred embodiments, together with the accompanying figures. Preferred embodiments are intended to explain and exemplify certain aspects of the present of invention but should not be construed as limiting its scope of protection.

### BRIEF DESCRIPTION OF THE DRAWINGS

The present invention and the following detailed description of preferred embodiments thereof may be better understood with reference to the following figures:

FIG. 1: Identification of putative HLA-DRA transcriptional regulatory elements. From the top, blue bars indicate the HLA-DRA gene body, orange and red bars indicate the regions with enhancer- and promoter-like signature by the ENCODE cCREs analysis, in pink are the peaks of histone modifications or RNA Polymerase II binding, and the gray bars on the bottom indicate the binding sites of specific transcription factors.

FIG. 2: Regulatory elements in the proximal HLA-DRA promoter region. A) schematic view of the regulome operating at the level of the HLA-DRA promoter; B) the output of MEME analysis, where the putative S-X-Y cassette is indicated as the orange-blue-red element. The corresponding DNA motif consensus is indicated in the bottom-left.

FIG. 3: human HEK293T cells transduced with increasing amount of hHLA-GFP. On the top of each dot plot is shown the amount of viral preparation used to transduce the target cells, expressed in ml. On the X-axis is plotted the GFP signal (FITC).

FIG. 4: In vivo GFP expression driven by the hHLA promoter. A) schematic view of the gating strategy used to determine the fraction of CD11b+, CD45.1+ and GFP+ cells in the brain-derived cells of the transplanted mice; b) Donor-cell chimerism (left), percentage of CD11b+CD45+/GFP+myeloid/microglial cells (center), and Mean of Fluorescence Intensity (MFI, right) in the whole set of three transplanted mice per vector-group.

### DETAILED DESCRIPTION OF PREFERRED EMBODIMENTS

In the following, several embodiments of the invention will be described. It is intended that the features of the various embodiments can be combined, where compatible. In general, subsequent embodiments will be disclosed only with respect to the differences with the previously described ones.

Unless defined otherwise, all technical and scientific terms used herein have the same meaning as commonly understood by those of ordinary skill in the art to which the invention belongs.

### Promoter Sequence

“Promoter” and “promoter sequence” are used interchangeably and refer to a DNA sequence capable of controlling the expression of a coding sequence or functional RNA. In general, a coding sequence is located 3′ to a promoter sequence. A promoter may be derived in its entirety from a native gene, may be composed of different factors derived from different promoters found in nature, or may contain synthetic DNA segments.

According to one aspect of the present invention there is provided a promoter sequence comprising or consisting of a polynucleotide sequence having SEQ ID NO: 1.

According to one aspect of the present invention there is provided a promoter sequence that shares more than 90%, preferably more than 95%, more preferably more than 98%, more preferably more than 99% identity with the sequence of SEQ ID NO:1. It is to be understood that different mutations in the promoter sequence may be introduced provided they do not impair the ability of the promoter to perform the functions described herein. “Percent (%) sequence identity” as used herein with respect to a reference polynucleotide sequence is defined as the percentage of nucleic acids in a candidate sequence that are identical to the nucleic acids in the reference polynucleotide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST.

According to one embodiment the promoter sequence is a synthetic sequence.

The invention further encompasses a mRNA sequence comprising or consisting of SEQ ID NO:2 or a mRNA sequence with more than 98%, more preferably more than 99% identity with the sequence of SEQ ID NO:2.

### Gene Transfer Vector/Lentiviral Vector

According to one aspect of the present invention there is provided a gene transfer vector for use in gene therapy comprising the promoter sequence of the invention.

In one embodiment the gene transfer vector comprises also the nucleotide sequence, which is a transgene, in particular comprising a transgene sequence, under the transcriptional control of said promoter sequence.

Expression vectors as described herein comprise regions of nucleic acid containing sequences capable of being transcribed. Thus, sequences encoding mRNA, tRNA and rRNA are included within this definition.

A vector is a tool that allows or facilitates the transfer of an entity from one environment to another. By way of example, some vectors used in recombinant DNA techniques allow entities, such as a segment of DNA (such as a heterologous DNA segment, such as a heterologous cDNA segment), to be transferred into a target cell. Optionally, once within the target cell, the vector may then serve to maintain the heterologous DNA within the cell or may act as a unit of DNA replication. Examples of vectors used in recombinant DNA techniques include plasmids, chromosomes, artificial chromosomes or viruses.

In particular, the term “plasmid”, as used herein, refers to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (e.g., bacterial plasmids having a bacterial origin of replication and episomal mammalian plasmids). Other vectors (e.g., non-episomal mammalian vectors) can be integrated into the genome of a host cell upon introduction into the host cell, and thereby are replicated along with the host genome. Certain plasmids are capable of directing the expression of genes to which they are operably linked.

Non-viral delivery systems include but are not limited to DNA transfection methods. Here, transfection includes a process using a non-viral vector to deliver a gene to a target mammalian cell.

In one embodiment the gene transfer vector is a viral vector, such as a lentiviral or adeno-associated viral vector, or it is a non-viral vector.

Suitable examples of non-viral gene transfer vectors that might be employed according to the present invention include all non-viral gene transfer vectors based on transposable elements, such as the sleeping beauty transposon system, or the PiggyBac transposon system.

In a preferred embodiment the gene transfer vector is derivable from a lentivirus, in particular is a lentiviral vector.

Within the context of this invention, a “lentiviral vector” means also a non-replicating vector for the transduction of a host cell with a transgene comprising cis-acting lentiviral RNA or DNA sequences, and requiring lentiviral proteins that are provided in trans. The lentiviral vector may be present in the form of an RNA or DNA molecule, depending on the stage of production or development of said retroviral vectors.

The lentiviral vector can be in the form of a recombinant DNA molecule, such as a plasmid. The lentiviral vector can be in the form of a lentiviral particle vector, such as an RNA molecule(s) within a complex of lentiviral and other proteins. Typically, lentiviral particle vectors, which correspond to modified or recombinant lentivirus particles, comprise a genome which is composed of two copies of single-stranded RNA. These RNA sequences can be obtained by transcription from a double-stranded DNA sequence inserted into a host cell genome (proviral vector DNA) or can be obtained from the transient expression of plasmid DNA (plasmid vector DNA) in a transformed host cell.

Lentiviral vectors derive from lentiviruses, in particular human immunodeficiency virus (HIV-1 or HIV-2), simian immunodeficiency virus (SIV), equine infectious encephalitis virus (EIAV), caprine arthritis encephalitis virus (CAEV), bovine immunodeficiency virus (BIV) and feline immunodeficiency virus (FN), which are modified to remove genetic determinants involved in pathogenicity and introduce new determinants useful for obtaining therapeutic effects.

It is to be understood that many different sources of lentiviral vectors can be used, and numerous substitutions and alterations in certain of the lentiviral vectors may be accommodated provided they do not impair the ability of the vector to perform the functions described herein.

In various embodiments, the lentiviral vector comprises the promoter sequence of the invention according to any of the embodiment herein disclosed.

In one embodiment, the gene transfer vector according to any of the embodiments described herein comprises one or more reporter gene sequences. As used herein, a “reporter gene sequence” is any gene sequence which, when expressed, results in the production of a protein whose presence or activity can be monitored. In one embodiment of the invention, the reporter gene sequence will encode an enzyme or other protein which is normally absent from the target cell, and whose presence can, therefore, definitively establish the presence of the vector in such a cell.

The invention further encompasses methods for producing a lentiviral vector particle. Such methods can comprise the steps of transfecting a suitable host cell with the lentiviral vector; transfecting the host cell with a packaging plasmid vector containing viral DNA sequences encoding at least structural and integrase proteins of a retrovirus; culturing said transfected host cell in order to obtain expression and packaging of said lentiviral vector into lentiviral vector particles; and harvesting the lentiviral vector particles resulting from the expression and packaging in said cultured host cells.

The invention further encompasses lentiviral vector particles comprising the lentiviral vector of the invention. The lentiviral vector particle can comprise a transgene sequence under control of the promoter of the invention. Preferably, the promoter sequence comprises a polynucleotide sequence that shares more than 90%, preferably more than 95%, more preferably more than 99% identity with the promoter sequence of SEQ ID NO:1.

The invention further encompasses a method for producing a lentiviral vector comprising introducing the set of DNA constructs herein disclosed into a host cell, and obtaining the viral vector particle, in one embodiment such method comprising the steps of:
a) transfecting a suitable host cell with the lentiviral vector according to any of the embodiment herein disclosed;
b) culturing said transfected host cell in order to obtain expression and packaging of said lentiviral vector into lentiviral vector particles; and
d) harvesting the lentiviral vector particles resulting from the expression and packaging of step c) in said cultured host cells.

The lentiviral vectors according to the invention can be directly administered to a patient through known routes of administration, including systemic, local, or cutaneous, intradermal, for instance intratumoral, administration routes. As used herein, the terms “administering,” “administration,” and the like refer to directly giving a patient a therapeutic agent (e.g., a lentiviral vector or a population of modified host cells of the invention) by any effective route.

Ex vivo administration, for instance ex vivo transduction of target cells followed by administration of the treated cells to the patient to be treated, is also encompassed by the invention.

### Transgene

Within the context of this invention, a “transgene” may be a nucleic acid sequence within a gene transfer vector, in particular a lentiviral vector that is not normally present or not correctly expressed in a cell to be transduced with the gene transfer vector, in particular a lentiviral vector. The lentiviral vector serves to introduce this sequence into the transduced cell. The term “transgene” does not include those sequences of the vector that facilitate transduction of the transgene. The transgene may be a sense or antisense nucleic acid molecule. In one embodiment of the invention, the transgene is an exogenous nucleic acid sequence. As used herein, the term “exogenous” describes nucleic acid sequence that is not found naturally in a particular organism (e.g., a human) or in a particular location within an organism (e.g., an organ, a tissue, or a cell, such as a human cell) or one that has been added to a cell, thus in the context of the present invention, the exogenous transgene is one that has been introduced to the cell or a parent precursor cell that the cell has arisen from (e.g. microglial cell from a modified HSPC).

According to a preferred embodiment of the invention, the transgene sequence encodes a polypeptide, in particular a therapeutic polypeptide or an enzyme, wherein said enzyme is selected for example from the group of lysosomal or catalytic enzymes, molecules involved in modulating inflammation or immune-response, a growth/trophic factor, a combination thereof or others.

According to some embodiments, said transgene encodes a polypeptide that provides curative, preventive, or ameliorative benefits to a subject diagnosed with or that is suspected of suffering from a pathological condition of the central nervous system (CNS), more in particular a pathological condition of the CNS characterized by neuroinflammation.

In one embodiment the promoter sequence herein disclosed is “operably linked” to the transgene. The term “operably linked” means that the components described are in a relationship permitting them to function in their intended manner.

### Host Cell

The invention further encompasses an isolated host cell comprising the promoter sequence or the gene transfer vector, in particular a lentiviral vector of the invention.

According to another aspect of the invention there is provided a cell infected or transduced with the gene transfer vector or particle according to the present invention. The cell may be transduced or infected in an in vivo or in vitro scenario, by using any approach known in the art.

If transduced or infected in vitro, for example, the desired recipient cells could be removed from a subject in need of therapy, transduced or infected with the gene transfer vector or particle according to the present invention, and reintroduced into the subject. Cells transduced in vitro can be screened for those cells harbouring the genes of interest using conventional techniques such as Southern blots and/or PCR, or by using selectable markers. Successfully transduced or infected cells should then be capable of expressing the introduced gene or other polynucleotide and/or the viral vector nucleic acid or part thereof in the cell.

Transduced or infected cells can be then formulated into pharmaceutical compositions, and the compositions introduced into the subject by various techniques as further described below, in one or more doses.

The cell may be derived from or form part of an animal, preferably a mammal, more preferably a human. The host cells according to any of the embodiments herein disclosed, can be particularly derived from a healthy individual or an individual with a disease to be treated, such as an individual with a diagnosed disease or disorder.

Cells suitable for the transduction/transfection and administration in the gene therapy methods of the invention include, but are not limited to stem cells, progenitor cells, and differentiated cells.

In one embodiment the cell is a hematopoietic stem cell or a hematopoietic progenitor cell. The term “hematopoietic stem cell” or “HSC” refers to multipotent stem cells having the capacity to self-renew and to differentiate into mature blood cells of diverse lineages, including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). In preferred embodiments, the cells are hematopoietic stem and/or progenitor cells isolated from bone marrow, umbilical cord blood, peripheral circulation (including mobilized peripheral blood), placental blood, foetal liver, and lymphoid soft tissue. In addition, HSCs also refer to long term repopulating HSC (LT-HSC) and short-term repopulating HSC (ST-HSC). LT-HSC and ST-HSC are differentiated, based on functional potential and on cell surface marker expression. Any of these HSCs can be used in any of the methods described herein.

Hematopoietic progenitor cells (HPCs) are descendants of HSCs that are capable of further differentiation to become specialised cells. Stem cells that self-renew produce more stem cells, those that start down the path of differentiation produce progenitor cells. A population comprising HSCs and/or HPCs are referred to herein as HSPCs.

The host cell could also be a microglia cell (i.e. transduced directly by in vivo gene transfer) or a monocyte/macrophage, also for extra-CNS applications. With the term “microglia”, as used herein, is meant an immune cell of the central nervous system.

The promoter according to any of the embodiments of the present invention is expected to be highly expressed also in circulating monocytes and in macrophages.

In particular embodiments, the transduced or transfected cells may be administered to a subject in need thereof according to the present invention by means of transplantation, e.g., as a part of a bone marrow transplant.

In one preferred embodiment, the invention provides transduced or transfected cells that have the potential to develop into brain microglial cells. In one embodiment, the cell is a myeloid or microglia cell derived from transplanted hematopoietic stem/progenitor cells.

According to a further aspect of the invention, there is provided also a method for producing a population of host cells, in particular hematopoietic stem cells, modified to express a transgene, particularly a transgene encoding a therapeutic polypeptide or enzyme, the method comprising contacting a sample of host cells with a gene transfer vector, viral particle, or composition according to any of the embodiments disclosed herein, under conditions to allow said vector, viral particle, or composition to transduce the host cells. Suitably, the method is carried out ex vivo.

In one embodiment, the above method further comprises a step of determining by means of qualitative and/or quantitative analysis the expression levels of said transgene in the transduced host cell. The terms “qualitative and/or quantitative analysis” are meant to encompass any standard technique or assays available in the art, which would be suitable for the determination and/or quantification of the expression levels of the transgene of interest. Non limiting examples of assays suitable for determining and/or quantifying the expression levels of any of the above-mentioned markers include an immunological assay, an aptamer-based assay, a histological or cytological assay, an RNA expression levels assay or a combination thereof. All tests indicated above are known to a person skilled in the art who, knowing the transgene whose expression has to be determined and/or quantified and the type of cells used, will be able to select the most suitable protocol.

In one preferred embodiment, transgene expression levels can be determined by measuring the concentration or relative abundance of a corresponding polypeptide and/or enzyme product encoded by the gene of interest. Protein expression assays suitable for use with the compositions and methods described herein include proteomic approaches, immunohistochemical and/or western blot analysis, immunoprecipitation, molecular binding assays, ELISA, enzyme-linked immunofiltration assay (ELIFA), mass spectrometry, mass spectrometric immunoassay, and biochemical enzymatic activity assays.

### Compositions

According to another aspect of the invention there is provided a pharmaceutical composition comprising the gene transfer vector or particle or host cell according to the present invention together with a pharmaceutically acceptable diluent, excipient or carrier. The invention further encompasses such compositions for use as a medicament, in particular in gene therapy, more in particular in ex vivo and/or in vivo gene therapy.

The present invention also provides a pharmaceutical composition for treating an individual by gene therapy, wherein the composition comprises a therapeutically effective amount of the vector of the present invention comprising one or more deliverable therapeutic and/or diagnostic transgenes(s) or a viral particle produced by or obtained from same. The pharmaceutical composition may be preferably for human usage. The composition may optionally comprise a pharmaceutically acceptable carrier, diluent, excipient or adjuvant.

As used herein, a “pharmaceutically acceptable carrier” includes any and all solvents, dispersion media, coatings, antibacterial and antifungal agents, isotonic and absorption delaying agents provided they are physiologically compatible, including pharmaceutically acceptable cell culture media. The choice of pharmaceutical carrier, excipient or diluent can be selected with regard to the intended route of administration and standard pharmaceutical practice. They can be injected for example parenterally, for example intra-cavernosally, intravenously, intra-parenchimally in the CNS, in the cerebrospinal fluid (intra-cerebral ventricular or intrathecal), intramuscularly or subcutaneously. For parenteral administration, the compositions may be best used in the form of a sterile aqueous solution which may contain other substances, for example enough salts or monosaccharides to make the solution isotonic with blood.

Sterile injectable solutions can be prepared by incorporating the active compounds of the invention in the required amount in the appropriate solvent with the various other ingredients enumerated above, as required, followed by filtered sterilization.

The skilled person can readily determine the amounts of gene vectors, viral particles or cells and optional additives, vehicles, and/or carriers in compositions to be administered. In particular, the amount of cells according to the present invention to be administered to a subject in need of therapy and the route of administration may depend on a number of factors such as, for example, on the expression level of the desired protein(s) in the cells, the percentage of successfully transduced cells, the vector copy number (VCN), the patient's age, body weight, sex the pharmaceutical formulation methods, as well as on the severity of the disease being treated.

The compositions should be stable under the conditions of manufacture and storage and should be preserved against the contaminating action of microorganisms, such as bacteria and fungi. The compositions can be presented in unit-dose or multi-dose sealed containers, such as ampules and vials.

If desired, the compositions of the invention may be administered in combination with other agents as well, such as, e.g., other proteins, polypeptides, small molecules or various pharmaceutically active agents. There is virtually no limit to other components that may also be included in the compositions, provided that the additional agents do not adversely affect the ability of the composition to deliver the intended gene therapy.

### Therapy

The invention further encompasses the gene transfer vector, the viral particle, the pharmaceutical composition, the host cell according to any of the embodiments herein disclosed for use as a medicament, in particular for use in a gene therapy, preferably for use in ex vivo and/or in vivo gene therapy, more preferably for use in hematopoietic stem/progenitor cell therapy. In particular for treating a pathological condition of the central nervous system (CNS), more in particular a pathological condition of the CNS characterized by neuroinflammation. In one particular embodiment, the gene transfer vector, the viral particle, or the pharmaceutical compositions according to any of the embodiments herein discloses could be used for in vivo transduction of microglia cell by means of intra-parenchymal or intra-intra-cerebral ventricular or intrathecal gene transfer.

The present invention contemplates that the gene transfer vectors, the viral particles, the pharmaceutical compositions, the host cells according to any of the embodiments described herein may also be used to prevent, and/or ameliorate a pathological condition of the CNS. The expression “ameliorate a pathological condition”, as used herein, means that the extent and/or undesirable clinical manifestations of the disease, disorder, or condition are lessened and/or the time course of the progression is slowed or lengthened, as compared to the extent or time course in the absence of treatment.

The invention further encompasses the gene transfer vector, the viral particle, the pharmaceutical composition, the host cell according to any of the embodiments herein disclosed for treating a disease selected from inherited or acquired neurodegenerative diseases (e.g. lysosomal storage disorders such as metachromatic leukodystrophy (MLD), globoid cell leukodystrophy (GLD), gangliosidosis GM1, gangliosidosis GM2, mucopolysaccharidoses (MPSs) etc., peroxisomal disorders such as X-linked adrenoleukodystrophy (X-ALD), adrenomyeloneuropathy (AMN) etc., adult onset acquired neurodegenerative conditions such as Alzheimer's disease (AD), amyotrophic lateral sclerosis (ALS), Parkinson's disease (PD), frontotemporal dementia (FTD), multiple sclerosis (MS) etc.).

The invention further encompasses also a method of treatment a subject comprising the step of administering in said subject the gene transfer vector, the viral particle, the pharmaceutical composition or the host cell according to any of the embodiments herein disclosed.

It is also herein disclosed a method of treatment of a subject comprising the following steps:
determining whether a subject is suffering from pathological condition of the central nervous system; and
if the subject is suffering from such condition, administering in said subject the gene transfer vector, the viral particle, the pharmaceutical composition or the host cell according to any of the embodiments herein disclosed.

In some embodiments, the subject undergoing treatment is the donor that provides cells (e.g. hematopoietic stem and/or progenitor cells) that are subsequently modified according to any of the previously described methods, before being re-administered to the subject. In such cases, withdrawn cells (e.g., HSPCs) may be re-infused into the subject following, for example, incorporation of a transgene encoding a therapeutic polypeptide or enzyme according to any one of the embodiments disclosed in the present specification. In cases in which the subject undergoing treatment also serves as the cell donor, the transplanted cells are less likely to undergo graft rejection.

Alternatively, the subject undergoing said treatment and the cell donor may be distinct.

It forms also part of the present invention the use in vitro of a gene transfer vector or a particle according to any of the embodiments described in the present specification, for regulating the expression of a transgene in a host cell, in particular in a hematopoietic stem cell or a hematopoietic progenitor cell. In one preferred embodiment, said gene transfer vector or particle are used to specifically upregulate the expression of said transgene in the presence of neuroinflammation and downregulate it under homeostatic CNS conditions in said host cell. The terms “upregulated”, as used herein, refer to an increased mRNA transcription level in comparison with the same expression generally determined in a healthy state and/or in comparison with the standard.

Having thus described different embodiments of the present invention, it should be noted by those skilled in the art that the disclosures herein are exemplary only and that various other alternatives, adaptations, and modifications may be made within the scope of the present invention. Accordingly, the present invention is not limited to the specific embodiments as illustrated herein.

Examples are reported below which have the purpose of better illustrating the embodiments disclosed in the present description, such examples are in no way to be considered as a limitation of the previous description and the subsequent claims.

### EXAMPLES

### Example 1—Design of a Novel Promoter Based on the Human HLA_DRA Putative Regulatory Region

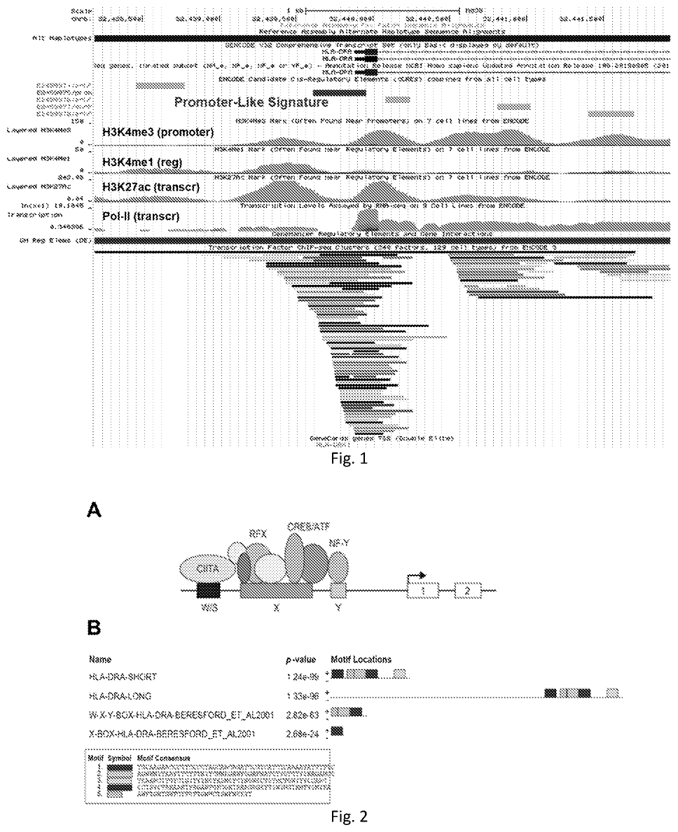

Several clusters of putative transcriptional regulatory elements were identified in the genomic region around the human HLA-DRA Transcriptional Start Site (TSS) on the chromosome 6, by integrating publicly available transcriptional and epigenetic data produced by the Broad Institute-Encode Project in human cells. Four active enhancers were identified as regions enriched in H3K4me1 and H3K27ac histone modifications and bound by hematopoietic-specific transcription factors as indicated by Chip-seq genomic maps (FIG. 1), and a region epigenetically marked as active promoter by peaks of H3K4me3, H3K27ac and Polymerase-II binding, coupled to evidences of active transcription by RNA-seq data. These genomic regions were additionally defined as Enhancer- and Promoter-Like Signature (orange and red horizontal bar in FIG. 1), by the ENCODE candidate Cis-Regulatory Elements (cCREs) analysis.

Once identified a broad region as putative HLA-DRA promoter, the corresponding genomic region by “The UCSC Genome Browser” (https://genome.ucsc.edu/) was obtained and analyzed in deep detail to identify key regulatory DNA motifs W/S-, X- and Y-box (FIG. 2A). Two sequences to be analyzed were defined, a short one consisting of the more proximal region to the HLA-DRA TSS (FIG. 2B, HLA-DRA-SHORT), or an extended version, including the peaks of epigenetic marks (FIG. 2B, HLA-DRA-LONG). These genomic sequences were scanned with MEME Suite to identify the basal S, X and Y boxes, using the sequences published in Beresford et al. as references (FIG. 2B). From the analysis, the HLA-DRA-SHORT sequence contained the whole W/S-X-Y cassette, and was chosen for an additional sequence optimization to generate a HLA-DRA-based synthetic promoter to be used in a lentiviral system to reproduce the proper HLA-DRA transcriptional regulation. The inventors extensively worked on the HLA-DRA-SHORT sequence to eliminate all the canonical and cryptic signals of poly-Adenylation, which could lead to premature stop of the lentiviral genome production in the packaging cells, and signals of splicing that may induce transcriptional alterations in the transduced cells by trans-splicing events occurring between the integrated provirus and the host genome. After the sequence optimizations, the final HLA-DRA-based synthetic promoter (hHLA) resulted 361 bp long.

### Example 2—Production of Lentiviral Vectors Carrying the New hHLA Promoter to Drive the GFP Expression

The hHLA promoter was synthesized by Genewiz (Leipzig, Germany), and cloned into a standard Self-Inactivating Lentiviral backbone to drive the expression of a Green Fluorescent Protein (GFP) to generate the LV.hHLA.GFP (hHLA-GFP). As control vector, an identical construct carrying a strong, constitutive human Phosphate Glycerate Kinase (PGK) promoter to drive the expression of the GFP was used (hPGK-GFP). Lentiviral particles were produced by 3rd generation lentiviral system by transient transfection of HEK293T cells following standard operating procedures. The LVs were then concentrated by ultracentrifugation, re-suspended in phosphate buffered saline (PBS) and stored at −80° C. until use. LV infectious titers were determined as Transducing Units per ml (TU/ml) of viral stock by evaluating the resulting Vector Copy Number (VCN) in HEK293T cells transduced with serial dilutions of each LV stock, after two weeks in culture. VCN was determined by digital droplet PCR (ddPCR). In several productions, all the LVs could be produced with an Infectious Titer generally above 109 TU/ml.

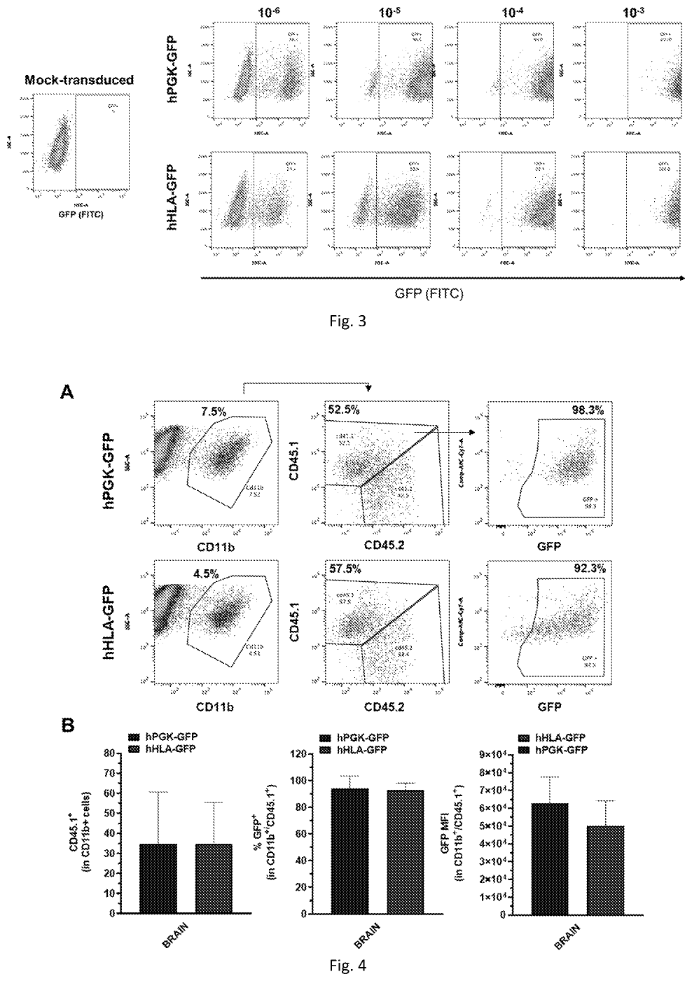

As shown in FIG. 3, the hHLA-GFP LV can efficiently transduce human HEK293T fibroblasts determining an increase of transgene expression at increasing amount of viral preparation.

### Example 3—Evaluation of the GFP Expression Driven by the hHLA Promoter In Vivo

The ability of the newly designed hHLA promoter to drive the expression of a transgene in vivo in the clinically relevant cell-type and organ, represented by microglia cells in the Central Nervous System (CNS), was investigated by a HSPC transplantation assay in wild-type C57BL/6J mice. After HSPC transplantation in fully-myeloablated recipient mice, these cells are able to repopulate the recipient CNS, replacing the original myeloid cell compartment with donor-derived myeloid/microglia-like cells.

Lineage-negative (Lin-) HSPCs were collected from the bone marrow (BM) of CD45.1 donor mice and first transduced with the hHLA-GFP or the hPGK-GFP vector, for 16 hours at an MOI of 100. After transduction, cells were washed and transplanted into fully-myeloablated CD45.2 recipient C57BL/6J mice (N=3 per LV) by intracerebroventricular (ICV) administration. Part of the cells were maintained in culture two weeks for VCN determination, which resulted comparable for the two LVs, at an average value above 10. Five days after ICV cell-administration, transplanted mice intravenously received total BM cells to support the systemic hematopoietic reconstitution.

Forty-five days post-transplantation, mice receiving HSPCs transduced with the LV hHLA-GFP (N=3) and mice receiving HSPCs transduced with the LV hPGK-GFP (N=3) were sacrificed. Brains were perfused, collected, and processed to a single cell homogenate to be analyzed for donor-cell chimerism and GFP expression by cytofluorimetry. Total myeloid/microglia cells were identified by CD45 and CD11b expression (FIG. 4A, left). Donor-cell chimerism was determined as the fraction of CD45.1+ versus CD45.2+ cells in the total CD11b+ cell population (FIG. 4A, center). Finally, the percentage of LV+ myeloid/microglia cells was determined by quantifying the GFP expression in the CD11bCD45.1+ cell population (FIG. 4A, right).

A comparable level of donor-cell chimerism was observed in the brain of mice transplanted with the two sets of Lin-cells, either transduced with the hHLA-GFP or the control LV. Similarly, the hHLA-GFP vector achieved a comparable transduction efficiency of the control LV in HSC-derived microglia cells (FIG. 4B, center). Finally, the hHLA promoter demonstrated to drive a high GFP expression (FIG. 4B, left), quantified as MFI, in microglia cells in vivo, slightly lower than the strong and constitutive hPGK promoter.

### Conclusions:

Here it was demonstrated that the novel hHLA promoter that was designed and developed, when inserted in a lentiviral vector to control the expression of a given transgene in immune-cell types, is able to induce gene expression, at mRNA and protein level, in vitro and in vivo, also in relevant CNS-associated myeloid/microglia-like cells derived from HSC transplantation. Indeed, this new hHLA promoter can induce a high transgene expression in vivo, in myeloid/microglia cells derived from transplanted HSPCs, suggesting its capability to maintain the correct regulatory profile upon pluripotent-cell differentiation to microglia cells, not being subjected to epigenetic silencing or other forms of undesired regulation.

These results strongly support the usage of this new hHLA promoter for therapeutic purposes, to determine a regulated expression of therapeutic or immune-modulatory molecules in gene and cell therapy approaches to treat pathological conditions of the CNS characterized by neuroinflammation.

hHLA promoter sequence
SEQ ID No: 1
tcgatttccgaattccactatccctgtctagaagtcagattggggttaa
agagtctgtccgtgattgactaacagtcttaaatacttgatttgttgtt
gttgttgtcctgtttgtttaagaactttacttctttatccaatgaacgg
agtatcttgtgtcctggaccctttgcaagaacccttcccctagcaacag
atgcgtcatctcaaaatatttttctgattggccaaagagtaattgattt
gcattttaatggtcagactctattacaccccacattctcttttctttta
ttcttgtctgttctgcctcactcccgagctctactgactcccaacagag
cgcccaagaagaaaacgc
corresponding mRNA sequence of hHLA promoter
SEQ ID No: 2
ucgauuuccgaauuccacuaucccugucuagaagucagauugggguuaa
agagucuguccgugauugacuaacagucuuaaauacuugauuuguuguu
guuguuguccuguuuuuuaagaacuuuuuuuuuugaacggaguaucuug
uguccuggacccuuugcaagaacccuuccccuagcaacagaugcgucau
cucaaaauauuuuucugauuggccaaagaguaauugauuugcauuuuaa
uggucagacucuauuacaccccacauucucuuuuuuuuauucuugucug
uucugccucacucccgagcucuacugacucccaacagagcgcccaagaa
gaaaacgc

1. A promoter sequence comprising or consisting of a polynucleotide sequence having SEQ ID NO: 1 or that shares more than 99% identity with the sequence of SEQ ID NO:1.

2. A gene transfer vector comprising the promoter sequence according to claim 1.

3. The gene transfer vector according to claim 2 wherein the vector is a viral or non-viral gene transfer vector.

4. The gene transfer vector according to claim 2, wherein said vector is a lentiviral vector or derivable from a lentivirus.

5. The gene transfer vector according to claim 1 comprising a transgene sequence, wherein said transgene sequence is under the transcriptional control of said promoter sequence.

6. The gene transfer vector according to claim 5 wherein said transgene codes for an enzyme, in particular wherein said enzyme is selected from the group of lysosomal or catalytic enzymes, molecules involved in modulating inflammation or immune-response, growth/trophic factor or combinations thereof.

7. A viral vector particle comprising the promoter sequence according to claim 1.

8. An isolated host cell comprising the promoter sequence according to claim 1.

9. An isolated host cell infected or transduced with the gene transfer vector of claim 2.

10. The cell according to claim 8 which is a hematopoietic stem cell or a hematopoietic progenitor cell.

11. A pharmaceutical composition comprising the gene transfer vector according to claim 2 and one or more excipients or diluents or carrier.

12. (canceled)

13. A method of ex vivo and/or in vivo gene therapy comprising administering the gene transfer vector of claim 2 to a subject in need thereof.

14. A method of hematopoietic stem therapy comprising administering the gene transfer vector of claim 2 to a subject in need thereof.

15. A method of treating a pathological condition of the central nervous system comprising administering the gene transfer vector of claim 2 to a subject in need thereof.

16. A method of treating a lysosomal storage disorders comprising administering the gene transfer vector os claim 2 to a subject in need thereof.

17. A method of regulating the expression of a transgene in a host cell comprising contacting the host cell with the gene transfer vector of claim 2.

18. The method of claim 16, wherein the lysosomal storage disease is metachromatic leukodystrophy (MLD), globoid cell leukodystrophy (GLD), gangliosidosis GM1, gangliosidosis GM2, mucopolysaccharidoses (MPSs) and other LSDs, peroxisomal disorders such as X-linked adrenoleukodystrophy (X-ALD), adrenomyeloneuropathy (AMN), and/or adult onset acquired neurodegenerative conditions such as Alzheimer's disease (AD), amyotrophic lateral sclerosis (ALS), Parkinson's disease (PD), or multiple sclerosis (MS).


SEQUENCE LISTING (2 sequences)

SEQ ID NO: 1 - DNA, 361 bp, Artificial Sequence
  SEQ ID No1-hHLA promoter sequence
tcgatttccgaattccactatccctgtctagaagtcagattggggttaaagagtctgtccgtgattgactaacagtcttaaatacttgatttgttgttgttgttgtcctgtttgtttaagaactttacttctttatccaatgaacggagtatcttgtgtcctggaccctttgcaagaacccttcccctagcaacagatgcgtcatctcaaaatatttttctgattggccaaagagtaattgatttgcattttaatggtcagactctattacaccccacattctcttttcttttattcttgtctgttctgcctcactcccgagctctactgactcccaacagagcgcccaagaagaaaacgc

SEQ ID NO: 2 - RNA, 361 nt, Artificial Sequence
  SEQ ID No2-corresponding mRNA sequence of hHLA promoter
ucgauuuccgaauuccacuaucccugucuagaagucagauugggguuaaagagucuguccgugauugacuaacagucuuaaauacuugauuuguuguuguuguuguccuguuuguuuaagaacuuuacuucuuuauccaaugaacggaguaucuuguguccuggacccuuugcaagaacccuuccccuagcaacagaugcgucaucucaaaauauuuuucugauuggccaaagaguaauugauuugcauuuuaauggucagacucuauuacaccccacauucucuuuucuuuuauucuugucuguucugccucacucccgagcucuacugacucccaacagagcgcccaagaagaaaacgc